(19) United States
(12) Patent Application Publication
(10) Pub. No.: US 2024/0055112 A1
(43) Pub. Date: Feb. 15, 2024

(54) GRADUAL SELF-SERVICE AND COMPLIANT AUTO-DIAGNOSTICS FOR MEDICAL DEVICES

(71) Applicant: KONINKLIJKE PHILIPS N.V., Eindhoven (NL)

(72) Inventors: Falk UHLEMANN, NORDERSTEDT (DE); Sebastiaan Jacobus Antonius DE HOOGH, OOSTERHOUT (NL); Sauvik BHATTACHARYA, EINDHOVEN (NL); Mauro BARBIERI, EINDHOVEN (NL); Tiblets Zeray DEMEWEZ, EINDHOVEN (NL)

(21) Appl. No.: 18/231,267

(22) Filed: Aug. 8, 2023

Related U.S. Application Data
(60) Provisional application No. 63/397,930, filed on Aug. 15, 2022.

(51) Int. Cl. G16H 40/40; G16H 40/20; G06N 20/00

(52) U.S. Cl. CPC G16H 40/40; G16H 40/20; G06N 20/00

(57) ABSTRACT

A service actions database stores credentials for performing respective service actions. A service engineer (SE) database stores credentials of respective SE's. In a method of authorizing a servicing session of a medical device, a service ticket to perform a servicing session of the medical device is received. Credentials of a candidate SE are retrieved from the SE database. One or more service actions are determined for resolving the service ticket. Credentials required to perform the servicing session are retrieved from the service actions database based on the determined one or more service actions. It is determined whether the credentials of the candidate SE satisfy the credentials required to perform the servicing session. An indication is output that the candidate SE is unqualified to perform the servicing session when the credentials of the candidate SE do not satisfy the credentials required to perform the servicing session.

### CROSS REFERENCE TO RELATED APPLICATIONS

This claims the benefit of U.S. Provisional Patent Application No. 63/397,930 filed Aug. 15, 2022. This application is hereby incorporated by reference herein.

### FIELD

The following relates generally to the medical device servicing arts, medical imaging device maintenance arts, medical imaging device maintenance training arts, and related arts.

### BACKGROUND

Service actions (e.g. preventive or corrective maintenance) are an integral part of the life cycle of complex computerized medical devices such as magnetic resonance imaging (MRI) scanners, computed tomography (CT) scanners, positron emission tomography (PET) scanners, or other medical imaging devices, radiation therapy (RT) devices, and so forth. Service activities are performed by a vendor of a medical device or authorized third party personnel or local biomedical engineers (“bio-meds”) depending on the service contract and the organizational structure of the customer of the medical device.

An organizational service setup identifying the person or entity who triggers and performs service actions at a customer site for specific equipment is often fixed. For example, magnetic resonance (MR) devices might always be serviced by a vendor of the device, while ultrasound US devices are always serviced by a third party at the customer site, regardless of the issue/required action.

This setup can often unnecessarily lead to long downtimes when the vendor or third party service is needed on site, high effort and cost for customers and the vendor to initiate and perform service actions, uncompliant self-made or local fixes, and so forth. Efforts like remote monitoring and its respective preventive/proactive/remote service actions try to mitigate the above issues but they cannot solve them when an (immediate) on-site action is required.

Downtime of a medical device can be minimized by ensuring a suitably qualified service engineer or other suitably qualified individual handles the repair. However, it may not be cost effective to have a repair handled by an overqualified individual if that particular repair could be handled by someone with less (but still adequate) training. Cost savings can tend to lead to assigning a lesser trained individual to handle a repair, but if that person is unable to complete the repair (i.e., the person is underqualified) then this leads to excess downtime as a more highly trained individual must then be called in. Unfortunately, it may not be immediately apparent whether a given individual is qualified to handle the repair.

The following discloses certain improvements to overcome these problems and others.

### SUMMARY

In some embodiments disclosed herein, a non-transitory computer readable medium stores a service actions database storing credentials for performing respective service actions, a service engineer (SE) database storing credentials of respective SE's, and instructions readable and executable by at least one electronic processor to perform a method of authorizing a servicing session of a medical device. The method includes: receiving a service ticket to perform the servicing session of the medical device; retrieving credentials of a candidate SE to perform the servicing session from the SE database; determining one or more service actions for resolving the service ticket; retrieving credentials required to perform the servicing session from the service actions database based on the determined one or more service actions; determining whether the credentials of the candidate SE satisfy the credentials required to perform the servicing session; and outputting an indication that the candidate SE is unqualified to perform the servicing session when the credentials of the candidate SE do not satisfy the credentials required to perform the servicing session.

In some embodiments disclosed herein, a non-transitory computer readable medium stores instructions executable by at least one electronic processor to perform a method of authorizing a servicing session of a medical device. The method includes: receiving a service ticket (40) to perform the servicing session of the medical device; determining whether credentials of a candidate service engineer (SE) to perform the servicing session satisfy credentials required to perform the servicing session; and outputting an indication that the SE is unqualified to perform the servicing session when the credentials of the SE do not satisfy the credentials required to perform the servicing session.

In some embodiments disclosed herein, an electronic servicing system is disclosed for servicing a medical device. The system includes an electronic processor programmed to: provide a log-in user interface (UI) for receiving user authentication information; provide a servicing UI; and perform an authenticated user qualification verification method on an authenticated user who enters valid user authentication information via the log-in interface. The authenticated user qualification verification method includes: retrieving a service ticket identifying a servicing session to be performed on the medical device; determining whether credentials of the authenticated user satisfy credentials required to perform the servicing session; in response to a determination that the credentials of the authenticated user do not satisfy the credentials required to perform the servicing session, preventing the authenticated user from accessing the servicing UI; and in response to a determination that the credentials of the authenticated user satisfy the credentials required to perform the servicing session, providing the authenticated user access to the servicing UI.

One advantage resides in reducing downtimes of medical devices.

Another advantage reside in reduced maintenance costs and efforts for servicing of medical devices.

Another advantage resides in ensuring servicing sessions of medical devices are performed by vendors or third party servicers who are qualified to do so.

Another advantage resides in minimizing time to resolve a service issue for a medical device.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an electronic servicing apparatus in accordance with the present disclosure.

FIG. 2 diagrammatically illustrates a method using the apparatus of FIG. 1.

### DETAILED DESCRIPTION

In software as a service (SAAS) or direct service settings, one entity may provide workflow efficiency support for servicing medical devices, while the actual servicing could in general be performed by engineers provided by the vendor of a medical device, or by hospital employees (e.g. bio-meds), or by a third party service provider, or various combinations thereof depending on the medical device needing service and the type of service that needs to be done on that medical device. The entity providing workflow efficiency support may, for example, provide a ticketing system via which medical personnel or other appropriate persons can request a service session. In another example, an automated failure prediction tool may apply modeling to log data and/or other automatically generated information in order to predict an upcoming component failure and proactively create a ticket for performing that service session. As an example, a model may be created to predict a time-to-failure of the filament of an X-ray tube of a computed tomography (CT) scanner, and when the predicted time-to-failure falls below some threshold a ticket to replace the X-ray tube is created.

When the ticket is received, the next task is to schedule the service session. This involves two components: identifying a time for the service session, and identifying a service person to perform the service action. The time can be determined in various ways such as by referencing a work schedule of the medical device or contacting the department/owner of the medical device to schedule the service session. Selecting a service person can be challenging, as various servicing tasks may require various training, certifications, or the like. If an overqualified service person is selected (for example, a service engineer provided by the vendor of the medical device) this may incur unnecessary cost as compared with having the service performed by a less-qualified (but still adequately qualified) individual such as an in-house biomed or other hospital employee, or a third-party service provider. On the other hand, if an underqualified service person is selected then the service session may fail to solve the problem, leading to additional medical device downtime and consequent additional cost. Embodiments disclosed herein efficiently and quickly validate that the service person is qualified to perform the service session before completing the scheduling of the service session with that service person assigned.

The following refers to servicing performed (or contemplated to be performed) by a duly trained/certified person as “compliant” servicing; while servicing performed (or contemplated to be performed) by a person without the requisite training/certification is “noncompliant.” The following discloses a system for ensuring compliant servicing.

A digital registry of service action is maintained, which stores service actions characterized by attributes such as device type(s), root cause, specific service actions, and so forth. The registry also stores training and/or certification requirements for each service action.

A medical devices database stores information about medical devices in the service workflow efficiency support program. The stored information is sufficient to associate medical devices with service actions from the registry.

A servicing personnel database stores information on servicing personnel enrolled in the support program (these could be vendor employees, but more relevantly can include local personnel such as bio-meds and third party service personnel). This database also stores the training and/or certifications of each enrolled individual.

A contracts database may also be provided to store contract types, validity, and expiration dates for contracting medical service providers (e.g. hospitals).

The support system receives service action requests, for example from an automated predictive maintenance algorithm operating on log data, and/or service requests filed by medical device operators or other personnel. The contracts database is consulted to determine available service options, and a service ticket is generated that is associated to the medical device.

When a service person is to execute a service ticket, the person logs into the system and identifies the ticket being serviced. The system consults the digital registry to determine the training/certification required to execute the ticket and compared this with training/certification of the service person retrieved from the servicing personnel database. If the person is determined to be qualified, then the person is authorized to proceed.

If, on the other hand, the person is determined to be unqualified, then the system could simply refuse authorization. In an optional addition, the system could arrange for the required training/certification. Depending on the type of training required, this could entail scheduling an in-person training session, or if the requisite training is available on demand, then the system could directly push the training to the person in real time to enable the person to become qualified to perform the service ticket immediately.

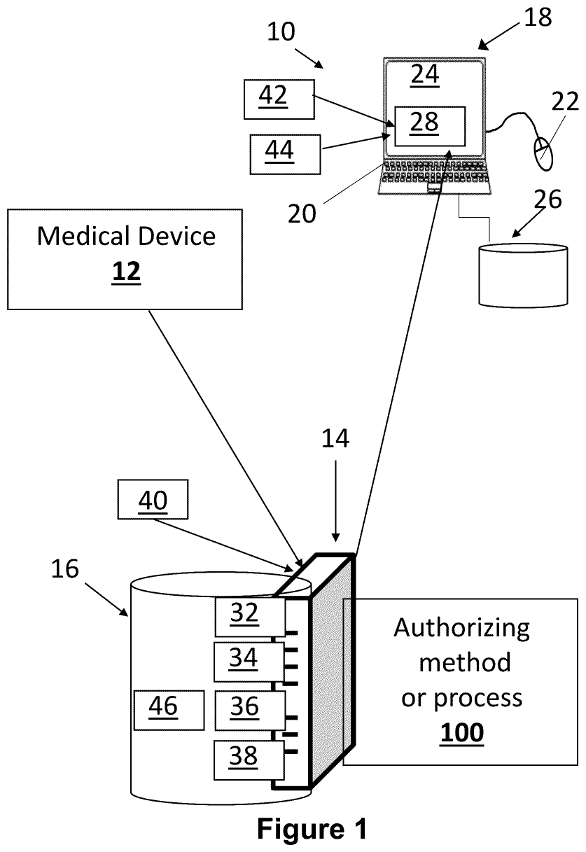

With reference to FIG. 1, an illustrative electronic servicing system or apparatus 10 for servicing an associated medical device 12 is shown. As shown in FIG. 1, the servicing apparatus 10 includes, or is accessible by, a server computer 14 typically disposed remotely from the medical device 12. The server computer 14 comprises a computer or other programmable electronic device that includes a non-transitory computer readable medium comprising a database 16.

An electronic processing device 18, such as a workstation computer, or more generally a computer, a smart device (e.g., a cellular telephone (“cell phone”), a smart tablet, and so forth), a controller of the medical device 12, or so forth is operable by a service engineer (SE). The electronic processing device 18 may also include a server computer or a plurality of server computers, e.g., interconnected to form a server cluster, cloud computing resource, or so forth, to perform more complex computational tasks. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18, or may include two or more display devices.

The electronic processor 20 is operatively connected with one or more non-transitory storage media 26. The non-transitory storage media 26 may, by way of nonlimiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The medical device 12, the server computer 14, and the electronic processing device 18 are typically in communication with each other via a communication link, which typically comprises an electronic network including the Internet augmented by local area networks for electronic data communications.

The database 16 of the server computer 14 can comprises a plurality of databases. For example, the database 16 of the server computer 14 can include a service actions database 32 storing credentials for performing respective service actions to service the medical device 12. Some nonlimiting illustrative examples of service actions include: replacement of a component or sub-component of the medical device 12; replacement of a consumable item of the medical device 12; various non-replacement service actions such as cleaning a component or sub-component, recalibrating a component or subcomponent, or so forth; software-based service actions such as installing new or updated software, configuring software, or so forth; performing a quality control (QC) assessment such as (in a case where the medical device 12 is a medical imaging device) operating the medical imaging device 12 to acquire images of a standard phantom and assessing image quality based on the acquired images; and/or so forth. These are merely illustrative examples. The credentials stored for each such service action may, by way of nonlimiting illustration, include: training credentials (e.g., the service person must have completed a specific training course to be qualified to perform the service action), certification credentials (e.g., the service person must have obtained a certain certification to be qualified to perform the service action); an academic degree credential (e.g., the service person must be a medical doctor or M.D. to perform the service action); professional license credential; and/or so forth. Some of these credentials may be specific to a particular make or model (or model series) of medical device, or to a class of medical devices (e.g. PET scanners), or otherwise limited. The content of the service actions database 32 are suitably constructed by the manufacturer of the medical device 12 and/or by another entity or entities with the expertise to identify appropriate credentials for performing each respective service action. For example, content of the service actions database 32 may be drawn up by senior service engineers working for the manufacturer of the medical device 12, possibly in consultation with legal experts who can provide information on any regulatory requirements such as tasks that might be considered “practice of medicine” and hence need to be performed by a medical doctor (M.D.) or electrical tasks that regulations require to be performed by a certified electrician, or so forth. These are merely nonlimiting illustrative examples.

A service engineer (SE) database 34 stores credentials of respective SE's to perform service actions to service the medical device 12. These may include, as some nonlimiting illustrative examples: a training record of the SE, certifications of the SE, academic degrees of the SE, professional licenses held by the SE, and/or so forth. A medical device database 36 can store data related to the medical device 12, such as medical device type data, potential issue root cause data, machine log data generated by the medical device 12, planned maintenance sessions for the medical device 12, and so forth. A contracts database 38 can store contracts for servicing the medical device 12, such as contract types, validity, expiration dates for contracting medical service providers, and so forth.

The apparatus 10 is configured as described herein to perform a method or process 100 of authorizing a servicing session of a medical device (i.e., the medical device 12), such as, for example an authenticated user qualification verification method or process 100. The database 16 stores instructions executable by the server computer 14 to perform the method 100. In some examples, the method 100 may be performed at least in part by cloud processing (that is, the server computer 16 may be implemented as a cloud computing resource comprising an ad hoc network of server computers).

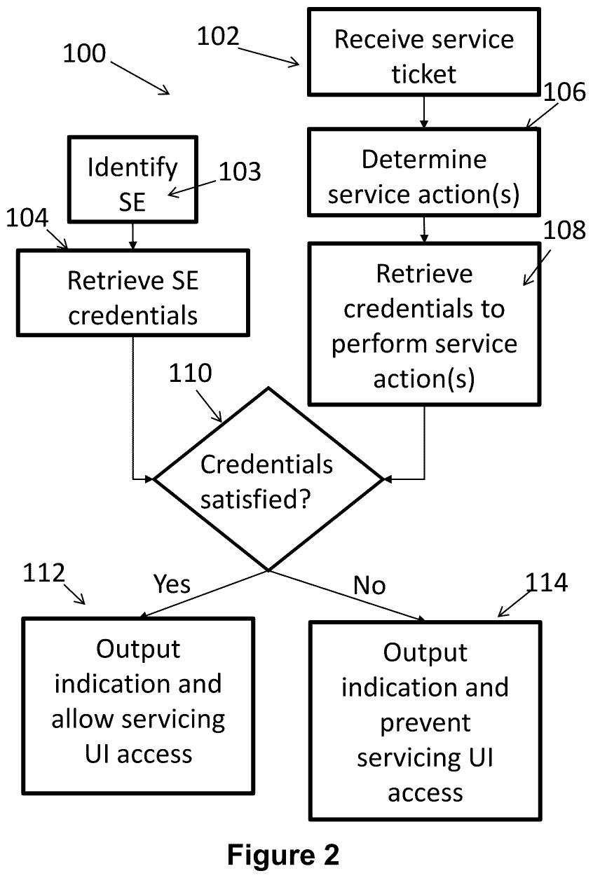

With reference to FIG. 2, and with continuing reference to FIG. 1, an illustrative embodiment of an instance of the method 100 is diagrammatically shown as a flowchart.

At an operation 102, the server computer 14 receives a service ticket 40 to perform a servicing session of the medical device 12. In some embodiments, the service ticket 40 can be automatically generated based on analysis of machine log data generated by the medical device 12, for example using a machine learning (ML) failure prediction model operating on machine log data generated by the medical device 12. In this case, the ticket may be generated for a proactive maintenance, such as automatically generating a service ticket to replace an X-ray tube before it fails based on a ML failure prediction model. In some embodiments, the service ticket 40 may be manually or semi-manually generated, for example by an electronic ticketing system at provides a standard form via which a medical professional or other user of the medical device 12 can enter information related to a service session being requested. In some embodiments, such an electronic ticketing system may be hosted by the server computer 14, in which case the server computer 14 may receive the service ticket 40 via the electronic ticketing system. The service ticket 40 includes information about the service session being requested, typically including at least the identity of the medical device 12 for which the service session is requested and information about a problem with the medical device 12 (either current or predicted) which is sought to be resolved by the service session being requested by the ticket.

At an operation 103, a candidate SE is identified for performing the service session. The candidate SE may be identified in various ways. In one approach, the candidate SE may attempt to log onto the electronic processing device 18 (which in this embodiment is a controller of the medical device 12) in a servicing user interface (UI) mode, and the login credentials entered thus identify the candidate SE. In another approach, a human user utilizes the electronic ticketing system (or a maintenance system, or other system running on the server 14 or elsewhere) to manually assign the candidate SE to handle the ticket. In yet another approach, an automated system (which again may be part of the electronic ticketing system or some other system running on the server 14 or elsewhere) may automatically assign the candidate SE based on a work schedule of an entity providing service engineers to the hospital for such tasks. These are merely nonlimiting illustrative examples.

At an operation 104, credentials of the candidate SE to perform the servicing session are retrieved from the SE database 34. The various servicing entities that supply service engineers to the hospital may include, for example: the hospital itself; the equipment vendor who manufactured the medical device 12 and provides servicing for the device 12 under a service contract; a third party service provider; or so forth. Each such servicing entity supplies the credentials of the SEs that work for that servicing entity to the SE database 34. Initial entry and subsequent timely updating of this information may be by way of a suitable user interface (UI) provided by the server computer 14 or other computing system in communication therewith. For example, the provider of the electronic servicing system 10 may maintain a web-based application (for example, on server 14) via which each servicing entity can log in using a browser to enter this information on its SEs.

At an operation 106, one or more service actions for resolving the service ticket 40 are determined, based at least on the content of the service ticket received at operation 102. In some embodiments, the service ticket 40 may directly specify the service action or actions to be performed. In other embodiments, the service ticket 40 may, by itself, be insufficient to directly determine the service actions to be performed. For example, the service ticket may merely identify symptoms observed by medical personnel when using the medical device 12. In this case, the service action determination operation 106 can include retrieving data related to the medical device 12 from the medical device database 36, and the one or more service actions for resolving the service ticket 40 are determined based on content of the service ticket 40 and the retrieved data related to the medical device 12. The operation 106 could also entail applying an electronic troubleshooting guideline to determine the service actions that are likely to be involved in resolving the service ticket 40. For example, if the service ticket 40 specifies the patient support is not moving the patient smoothly, a corresponding troubleshooting guideline may comprise a decision tree whose root includes this symptom and has nodes corresponding to service actions for resolving this type of problem such as oiling gearing of the patient support, replacing a belt or belts of the patient support, replacing one or more sub-components of the patient support, and/or so forth, and these nodes thus correspond to service actions. In some such embodiments, if the electronic troubleshooting guideline provides information on probabilities that various nodes will be traversed, this information can be used in operation 106 to select the service actions required to resolve the ticket 40 as the most probable service actions, or as those service actions whose probability is above some threshold, or so forth. As yet another contemplated embodiment, the operation 106 could be semimanual. For example, the service ticket 40 might be reviewed by a remote service engineer (RSE) or the like who then manually identifies which service action or actions are likely to be involved in resolving the ticket 40.

In some embodiments, the operation 106 could entail cross-correlating active tickets to schedule a single (or joint) servicing session to handle two (or more) cross-correlated open tickets. The cross-correlation may be based on locational factors, such as the tickets being for the same medical imaging device (thereby reducing downtime for the imaging device and travel costs for a field service engineer who can handle both tickets in one trip), or the tickets being for different medical imaging devices at the same location (thereby reducing travel costs for a field service engineer who can handle both servicing sessions in a single trip), or the tickets being for different medical imaging devices located in a common geographical area (again reducing travel costs). In the lattermost example, a traveling salesman-type optimization can optionally also be performed to schedule the servicing sessions for the imaging devices along an efficient route. In other embodiments, the cross-correlation may be based on similarity of the service tasks involved, thereby enabling similar tasks to be grouped and assigned to a single service engineer to maximize efficiency. These are merely illustrative examples.

It will be appreciated that the operations 104 and 106 can be performed simultaneously or sequentially in either suitable order.

At an operation 108, credentials required to perform the service actions identified in the operation 106 as being (or likely being) involved in resolving the servicing session identified by the ticket 40 are retrieved from the service actions database 32. At an operation 110, the server computer 14 is configured to determine whether the credentials of the candidate SE satisfy the credentials required to perform the servicing session. In some embodiments, the credential determination operation 108 can include retrieving, from the contracts database 38, contract data related to contracts for servicing the medical device 12, and the service action(s) for resolving the service ticket 40 are further determined based on the contract data. The contract data may include, for example, contract types, validity, and expiration dates for contracting medical service providers.

At an operation 112, when the credentials of the SE satisfy the credentials required to perform the servicing session, an indication 42 is output on the display device 24 that the SE is qualified to perform the servicing session. In some embodiments, in which the operation 103 identifies the candidate SE based on that person attempting to log into the servicing mode of the medical device 12, the log-in user interface (UI) for receiving user authentication information and a servicing UI are provided on the GUI 28. If the user is identified as an SE satisfying the credentials required to perform the servicing session, then the candidate SE is provided access to the servicing UI (possibly without an explicit indication that the candidate SE has been determined to be qualified to perform the servicing session—rather, such acknowledgment is implicit in the fact that the SE is permitted to log in.

However, when the credentials of the candidate SE do not satisfy the credentials required to perform the servicing session, then, at an operation 114, an indication 44 is output on the display device 24 that the candidate SE is unqualified to perform the servicing session. An unqualified candidate SE can be identified based on the candidate SE attempting to log onto an electronic servicing system (such as a service mode for the imaging device 12) for servicing the medical device 12 via the log-in UI. The indication 44 that that the candidate SE is unqualified to perform the servicing session when the credentials of the candidate SE do not satisfy the credentials required to perform the servicing session comprises outputting a refusal to permit the SE to log onto the electronic servicing system for servicing the medical device 12, and the user is prevented from accessing the servicing UI.

In some examples, when the candidate SE unqualified to perform the servicing session, the SE can schedule, via the GUI 28, a training session for the SE to obtain the credentials to perform the servicing session. In another example, training materials 46 (stored in the database 16) can be provided on the electronic processing device 18 for the SE to obtain the credentials to perform the servicing session.

In other embodiments, in which the candidate SE is assigned to the service ticket 40 further upstream, for example by the candidate SE being assigned to the ticket 40 as the service session is being scheduled, the output 112 or 114 can be provided via the UI through which the service session is being scheduled. Here, if the candidate SE is unqualified, in the operation 114 it may be useful to provide on the display of the UI an indication of the missing qualifications of the candidate SE, so that these can be remediated ahead of the scheduled servicing session by suitable training of the candidate SE, or to advise and assist the human scheduler in selecting another suitably qualified SE. These are merely nonlimiting illustrative examples.

As another contemplated variant, again most suitable in the case of upstream application of the method 100, the operation 110 may also make a determination as to whether the candidate SE is overqualified. This can be based on information explicitly contained in the service actions database 32 (e.g., identifying both minimum and maximum qualifications for certain service actions) or can be based on an implicit determination (e.g., if the candidate SE possesses credentials not expected to be utilized in resolving the ticket 40). If the candidate SE is overqualified, then the candidate SE is not prevented from performing the servicing session; however, the UI may notify of this overqualification to enable the human scheduler to consider assigning a less qualified (but still adequately qualified) SE to the ticket 40 in the interest of reducing cost of resolving the ticket 40.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
a service actions database storing credentials for performing respective service actions;
a service engineer (SE) database storing credentials of respective SE's; and
instructions executable by at least one electronic processor to perform a method of authorizing a servicing session of a medical device, the method comprising:
receiving a service ticket to perform the servicing session of the medical device;
retrieving credentials of a candidate SE to perform the servicing session from the SE database;
determining one or more service actions for resolving the service ticket;
retrieving credentials required to perform the servicing session from the service actions database based on the determined one or more service actions;
determining whether the credentials of the candidate SE satisfy the credentials required to perform the servicing session; and
outputting an indication that the candidate SE is unqualified to perform the servicing session when the credentials of the candidate SE do not satisfy the credentials required to perform the servicing session.

2. The non-transitory computer readable medium of claim 1, wherein determining the one or more service actions for resolving the service ticket includes:
retrieving data related to the medical device from a medical device database; and
determining the one or more service actions for resolving the service ticket based on content of the service ticket and the retrieved data related to the medical device.

3. The non-transitory computer readable medium of claim 2, wherein the data related to the medical device includes one or more of medical device type data, potential issue root cause data, and/or machine log data generated by the medical device.

4. The non-transitory computer readable medium of claim 2, wherein determining whether the credentials of the SE satisfy the credentials required to perform the servicing session includes:
retrieving, from a contracts database, contract data related to contracts for servicing the medical device;
wherein the determining of one or more service actions for resolving the service ticket is further based on the retrieved contract data.

5. The non-transitory computer readable medium of claim 1, wherein the receiving of the service ticket includes:
automatically generating the service ticket for proactive maintenance based on output of a machine learning (ML) failure prediction model operating on machine log data generated by the medical device.

6. The non-transitory computer readable medium of claim 1, wherein the determining of the one or more service actions for resolving the service ticket includes:
identifying a related service ticked by cross-correlating the related service ticket with the service ticket;
wherein the one or more service actions are determined based on both the service ticket and the related service ticket.

7. The non-transitory computer readable medium of claim 1, wherein the method further comprises:
identifying the candidate SE based on the candidate SE attempting to log onto an electronic servicing system for servicing the medical device;
wherein the indication that the candidate SE is unqualified to perform the servicing session when the credentials of the candidate SE do not satisfy the credentials required to perform the servicing session comprises outputting a refusal to permit the SE to log onto the electronic servicing system for servicing the medical device.

8. The non-transitory computer readable medium of claim 1, wherein the method further includes:
outputting an indication that the SE is qualified to perform the servicing session when the credentials of the SE satisfy the credentials required to perform the servicing session.

9. The non-transitory computer readable medium of claim 8, wherein the indication that the candidate SE is unqualified to perform the servicing session when the credentials of the candidate SE do not satisfy the credentials required to perform the servicing session includes:
providing a user interface via which the candidate SE can schedule a training session for the SE to obtain the credentials to perform the servicing session.

10. The non-transitory computer readable medium of claim 8, wherein the indication that the candidate SE is unqualified to perform the servicing session when the credentials of the candidate SE do not satisfy the credentials required to perform the servicing session includes:
providing training materials on an electronic processing device operable by the SE for the SE to obtain the credentials to perform the servicing session.

11. A non-transitory computer readable medium storing instructions executable by at least one electronic processor to perform a method of authorizing a servicing session of a medical device, the method comprising:
receiving a service ticket to perform the servicing session of the medical device;
determining whether credentials of a candidate service engineer (SE) to perform the servicing session satisfy credentials required to perform the servicing session; and
outputting an indication that the SE is unqualified to perform the servicing session when the credentials of the SE do not satisfy the credentials required to perform the servicing session.

12. The non-transitory computer readable medium of claim 11, wherein determining the one or more service actions for resolving the service ticket includes:
retrieving data related to the medical device from a medical device database; and
determining the one or more service actions for resolving the service ticket based on content of the service ticket and the retrieved data related to the medical device.

13. The non-transitory computer readable medium of claim 12, wherein the data related to the medical device includes one or more of medical device type data, potential issue root cause data, and/or machine log data generated by the medical device.

14. The non-transitory computer readable medium of claim 12, wherein determining whether the credentials of the SE satisfy the credentials required to perform the servicing session includes:
retrieving, from a contracts database, contract data related to contracts for servicing the medical device;
wherein the determining of one or more service actions for resolving the service ticket is further based on the retrieved contract data.

15. The non-transitory computer readable medium of claim 14, wherein the contract data includes contract types, validity, and expiration dates for contracting medical service providers.

16. The non-transitory computer readable medium of claim 11, wherein the method further comprises:
automatically generating the service ticket based on analysis of machine log data generated by the medical device.

17. The non-transitory computer readable medium of claim 11, wherein the method further comprises:
identifying the candidate SE based on the candidate SE attempting to log onto an electronic servicing system for servicing the medical device;
wherein the indication that the candidate SE is unqualified to perform the servicing session when the credentials of the candidate SE do not satisfy the credentials required to perform the servicing session comprises outputting a refusal to permit the SE to log onto the electronic servicing system for servicing the medical device.

18. The non-transitory computer readable medium of claim 11, wherein the method further includes:
outputting an indication that the SE is qualified to perform the servicing session when the credentials of the SE satisfy the credentials required to perform the servicing session.

19. An electronic servicing system for servicing a medical device, the system comprising an electronic processor programmed to:
provide a log-in user interface (UI) for receiving user authentication information;
provide a servicing UI; and
perform an authenticated user qualification verification method on an authenticated user who enters valid user authentication information via the log-in interface, the authenticated user qualification verification method including:
retrieving a service ticket identifying a servicing session to be performed on the medical device;
determining whether credentials of the authenticated user satisfy credentials required to perform the servicing session;
in response to a determination that the credentials of the authenticated user do not satisfy the credentials required to perform the servicing session, preventing the authenticated user from accessing the servicing UI; and
in response to a determination that the credentials of the authenticated user satisfy the credentials required to perform the servicing session, providing the authenticated user access to the servicing UI.

20. The electronic servicing system of claim 19, wherein the authenticated user qualification verification method further includes:
in response to a determination that the credentials of the authenticated user do not satisfy the credentials required to perform the servicing session, providing the authenticated user with training materials via which the authenticated user can obtain the credentials to perform the servicing session or access to a scheduling system for scheduling a training session via which the authenticated user can obtain the credentials to perform the servicing session.
